(19) United States
(12) Patent Application Publication
(10) Pub. No.: US 2022/0143317 A1
(43) Pub. Date: May 12, 2022

(54) CARTRIDGE BASED AUTO-INJECTOR

(71) Applicant: New Injection Systems Ltd., Stowmarket (GB)

(72) Inventor: Stephen DUNNE, Stowmarket (GB)

(73) Assignee: New Injection Systems Ltd., Stowmarket (GB)

(21) Appl. No.: 17/431,586

(22) PCT Filed: Feb. 21, 2020
(86) PCT No.: PCT/EP2020/054704

§ 371 Date: Aug. 17, 2021

(30) Foreign Application Priority Data
Feb. 19, 2019 (GB) 1902355.5

(51) Int. Cl. A61M 5/24; A61M 5/20; A61M 5/32; A61M 5/315; A61L 2/20

(52) U.S. Cl. CPC A61M 5/2459; A61L 2101/44; A61M 5/2033; A61M 5/3297; A61M 5/3204; A61M 5/3234; A61M 5/2046; A61M 5/3157; A61L 2/206; A61L 2/20; A61M 2005/2407; A61M 2207/00; A61L 2202/24; A61L 2202/122; A61M 5/2422

(57) ABSTRACT

The present invention is an auto-injector for the administration and self-administration of drugs by subcutaneous, intramuscular or intra-dermal injection and for drug solutions or suspensions. The auto-injector may comprise three modules for ease of assembly, the modules principally including a standard cartridge module with stopper and septum, a power source and indicator module that pushes against the cartridge stopper pressurizing contents, an injection progress indicator and a needle shuttle comprising of two needles, one needle being the injection needle and one needle being the septum needle. A needle guard ensures that the needle is always covered thus hiding the needle before and after the injection and that prevents needle stick injurie.

The present invention is an auto-injector for the administration and self-administration of drugs by subcutaneous, intramuscular or intra-dermal injection and for drug solutions or suspensions. This application describes an injection system that may be used by the patient for self-injection or by a medical professional.

Auto-injectors automate the injection stroke alleviating the need for the patient or professional to actuate the plunger rod.

The auto-injector according to the present invention comprises in an outer casing that holds a standard cartridge comprising a barrel with a drug contents being contained by a movable plunger or stopper at one end, and a seal or septum and metal crimp or cap to hold said septum in place at the other end.

A biased means that pushes against the movable plunger or stopper of the cartridge thus pressurizing the drug contents. A needle shuttle is held within casing comprises two needles that are attached and connected to each other, the first needle being the injection needle and the second needle being the septum needle. A means or shoulder retains the cartridge in its correct position when inserted and prevents it from being pushed against the needle shuttle. A needle guard is connected to the casing, being biased outwards by a spring or the like such that the needle is always covered by the needle guard, thus hiding the needle before and after the injection.

The auto-injector according the present invention may further comprise a needle guard locking mechanism that prevents the needle guard from being pushed back a second time, thus preventing needle stick injuries after use.

The auto-injector may further comprise an indicator. The indicator may be in form of an indicator rod that is attached to the biased means or the movable plunger or stopper and thus moves with it, thus allowing to indicate the progress of the injection progress. The indicator rod may be connected and used to operate any other indicator such as a rotary indicator or an electronic indicator or a connectivity device.

In one embodiment, the indicator extends beyond the rear casing to a distance determined by the fill volume and enabling the user to monitor the status of the injection.

In a preferred embodiment, the indicator rod has a closed end and contains the spring within. The outer casing comprises a portion that allows to monitor the progress and status of the injection progress, for example a cap which is transparent or has viewing windows. If the drug volume is fully injected, the indicator is no longer visible indicating end of injection. Placing the spring inside the indicator has the advantage that the spring can be longer than if the indicator is placed inside the spring or the device may be shorter. A longer spring means a higher mean force which is advantageous for viscous drugs.

The invention further relates to a modular designed auto injector which is composed of several sub-assemblies or modules. For ease of assembly, the modular auto-injector comprises three sub-assemblies, namely a cartridge, a power source and indicator sub-assembly and a needle and needle guard sub-assembly.

The cartridge sub-assembly comprises a barrel, a drug volume that is bounded by a plunger and a crimped septum.

The power source and indicator assembly comprises a biased means or spring and an indicator rod that is held within a rear casing. The invention allows for the cartridge to have a variable fill volume when assembled with the power source and indicator sub-assembly.

In order to indicate the status and progress of the injection process, the indicator rod may be connected to either the biased means or spring or to the movable plunger or stopper upon assembly of the device in a way that it moves with it.

If the biased means is a spring it may further comprise a spring locking member. In this case the power source and indicator assembly may further comprise a second weaker spring to pressurize the cartridge drug contents prior to injection while the first spring is locked by the spring locking member. The second spring also avoids a pressure pulse when the second spring is released.

The needle and needle guard sub-assembly comprises a front casing holding a needle shuttle with an injection needle and a septum needle, the shuttle being held within the casing a needle guard for preventing needle stick injuries that is biased outwards by a spring, a locking mechanism that prevents the needle guard from being pushed back a second time, and a means to retain the cartridge in its correct position when inserted.

The injection needle and the septum needle may have the same diameter or gauge or have different diameters or gauges. They may have the same type of bevel tip or different types of bevel tip. For example, to minimize injection time the septum needle may be of larger diameter or smaller gauge than the injection needle. For simplicity a single needle may be used with two sharp ends or bevels. Different needle gauges and/or bevels can be used to ensure the injection needle penetrates the injection site before the septum needle penetrates the septum to ensure a full injection. The interchange of the injection needle allowing adaption for subcutaneous, intramuscular or intra-dermal injection.

The modular auto-injector may be assembled by an easy two-step process such as placing the cartridge into the needle and needle guard sub-assembly followed by connecting it with the power source and indicator sub-assembly in a way that the drug contents in the cartridge are pressurized. If the device is intended for single use, any connection means to lock the sub-assemblies of the modular auto-injector together may be used, such as a snap fit.

In a preferred embodiment, the modular auto-injector can be re-assembled or is multiple use. In this case the needle and needle guard assembly and the power source and indicator assembly may be connected by a a bayonet fit or a screw or are hinged together to allow a reversible connection. In addition, the biased means of the power source and indicator assembly allows for a multiple use such as an electric motor, a liquified gas canister or a spring with means to re-compress the spring. Finally, the needle and needle guard assembly comprises a seal and a removable needle cap that always keep the needles and the cartridge septum sterile.

The embodiments of the auto-injector according to the present invention may be assembled under sterile conditions or may be submitted to terminally sterilization after assembly.

If the drug in the cartridge is a small molecule (non-biologic) the assembled device may be terminally sterilized in a blister or pouch using gas, vapor or radiation such as gamma ray, e-beam or any other.

When sterilizing the device with sensitive drug contents such as biologic drugs, the blister/pouch with the assembled device has at least a portion made from gas permeable material. The device may have gas pathways to assure that the needles and the external surface of the cartridge septum are sterilized. The device can then be placed in a gaseous environment such as ethylene oxide or nitrogen dioxide for sterilization.

To prevent the gas affecting sensitive drug contents, the cartridge assembly may preferably have gas impermeable components, in particular plunger/stopper and septum/cap. The gas used should preferably not be at elevated temperatures and preferably below body temperature and preferably below 36° C.

The device may be supplied as a ready-to-use medical device or supplied pre-assembled as a combination product (“kit”) for self-assembly by the user.

Sterilisation may take place before assembly or after assembly in which case it is terminally sterilized. If sterilized before assembly only the needle and guard sub-assembly or module 182 need be sterilized. The assembled device may be terminally sterilized in a blister or pouch. A needle cap and maybe a seal is required if the device is sterilized outside a blister. If the device is assembled by the user, the cartridge can be supplied in a blister or with no blister in which case the septum needs to be wiped with ethanol before device assembly.

Table 1 shows an overview of the different options as regards sterilization, options 3 to 6 allowing for re-assembly.

TABLE 1

| | | | | Gas permeable |
|---|---|---|---|---|
| | | Seal 128 | Cap 126 | elastomer |
| | | essential? | essential? | essential? |
| 1. | Fully assembled | NO | NO | N/A |
| | with cartridge | | | |
| | IN BLISTER | | | |
| 2. | Fully assembled | NO | YES | Yes for gas |
| | with cartridge | | | No for radiation |
| | OUTSIDE BLISTER | | | |
| 3. | Sub-assembly 182 | NO | NO | N/A |
| | IN BLISTER | | | |
| 4. | Sub-assembly 182 | YES | YES | Yes for gas |
| | OUTSIDE BLISTER | | | No for radiation |
| 5. | Sub-assembly 182 | NO | NO | N/A |
| | assembled WITH | | | |
| | cartridge 181 | | | |
| | IN BLISTER | | | |
| 6. | Sub-assembly 182 | NO | YES | Yes for gas |
| | assembled WITH | | | No for radiation |
| | cartridge 181 | | | |
| | OUTSIDE BLISTER | | | |

The auto-injector according to the present invention may be used with drug suspensions or solutions and API's or biologics. If the suspension is unstable with settling tendencies the cartridge needs to be shaken before use to re-mix.

The modular design has the advantage that each sub-assembly may be produced separately under conditions individually adapted. It further allows for flexibility in production and allows different auto injectors to share modules. The modular design also allows for part exchangeability which bears the additional advantage is that modules or sub-assemblies can be interchanged to change the characteristics of the auto-injector. For instance, by changing the needle and hub and needle guard assembly the device can be changed from a subcutaneous auto-injector to an intramuscular or intra-dermal auto-injector. It also allows for the power source module to be a reusable device. The modular designed auto-injector does also allow the cartridge to be exchanged, either after it has been used or if its shelf-life has expired.

The present invention allows for the use of industry standard injection cartridge and manufacture auto-injectors with a dry needle during storage and with various other advantages.

With reference to the figures, specific embodiments of the present invention are described hereinafter:

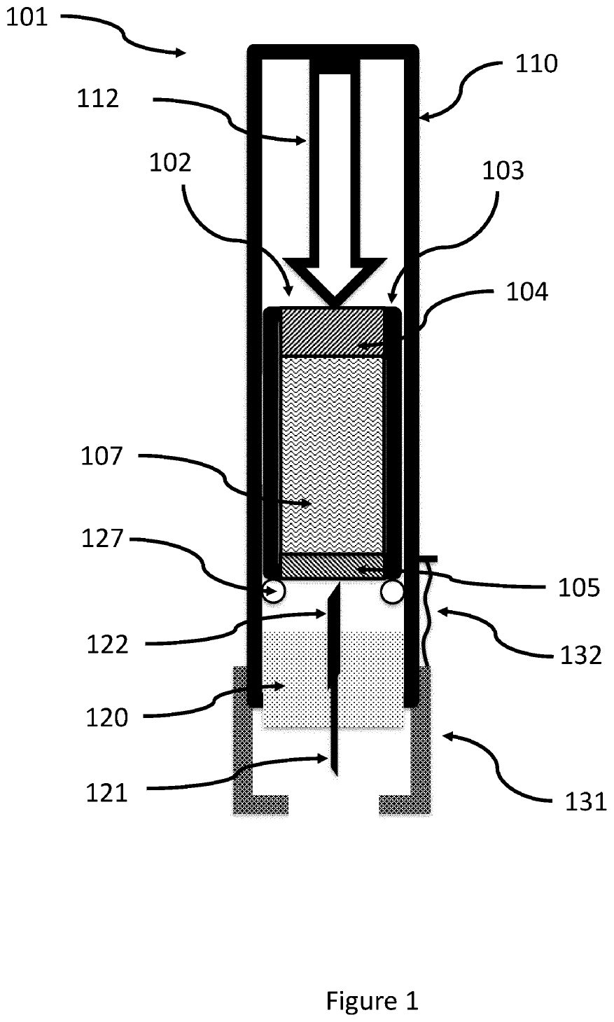

FIG. 1 shows an auto injector 101 according to the present invention. A standard cartridge 102 has barrel 103 with drug solution or suspension 107 within contained by a movable plunger or stopper 104 at one end and a rubber seal or septum and metal crimp 105 at the other end. The cartridge 102 is held in casing 110. A biased means 112 pushes against the stopper 104 pressurizing contents 107. A needle shuttle 120 is held within casing 110. Shuttle 120 has two needles 121 and 122 attached and connected to each other. Needle 121 is the injection needle and needle 122 is the septum needle. A means 127 retains cartridge 103 in its correct position when inserted and prevents it from being pushed against the shuttle. A needle guard 131 and spring 132 are held to casing 110. Needle guard 131 has the purpose of preventing needle stick injuries after injection.

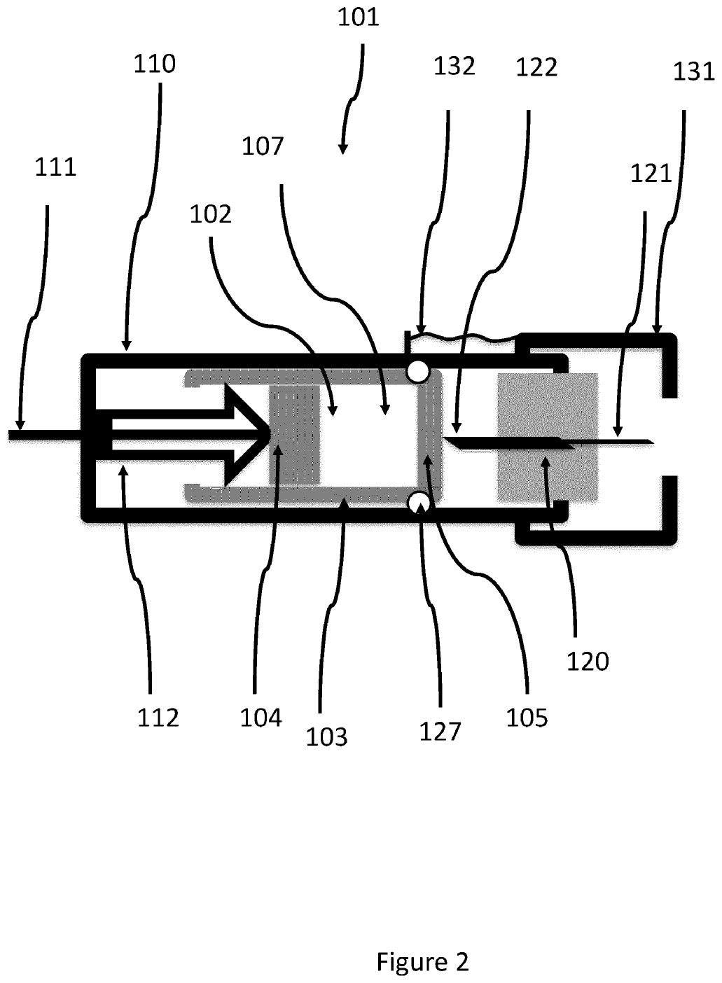

FIG. 2 shows the auto injector 101 with an indicator rod 111 being attached to stopper 104.

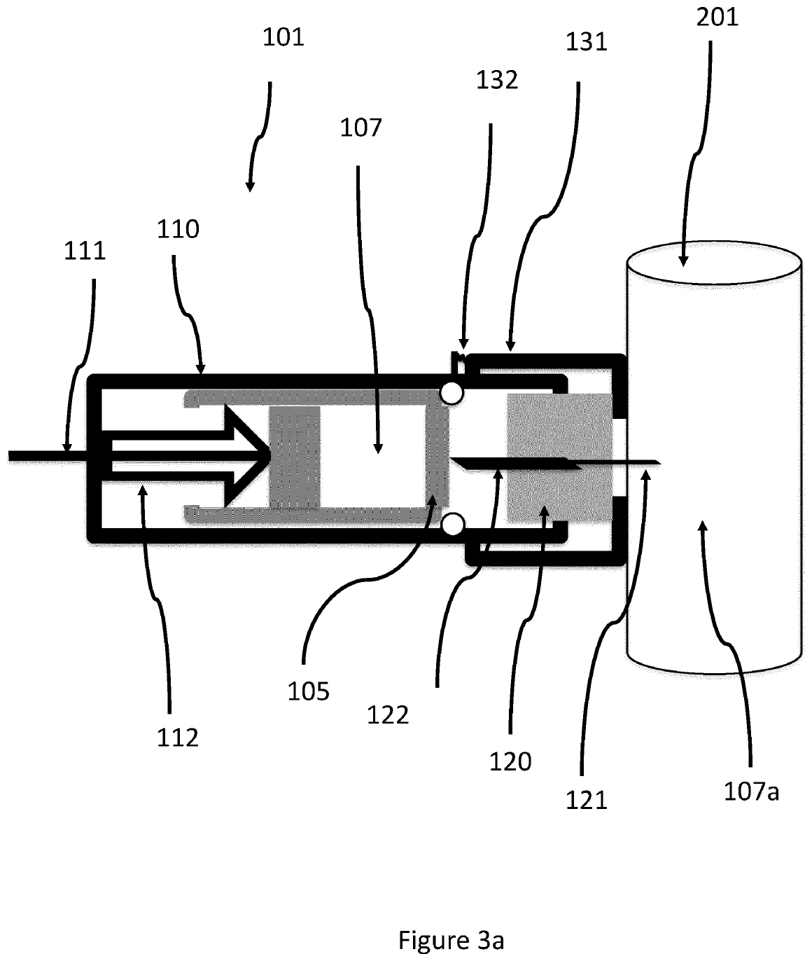

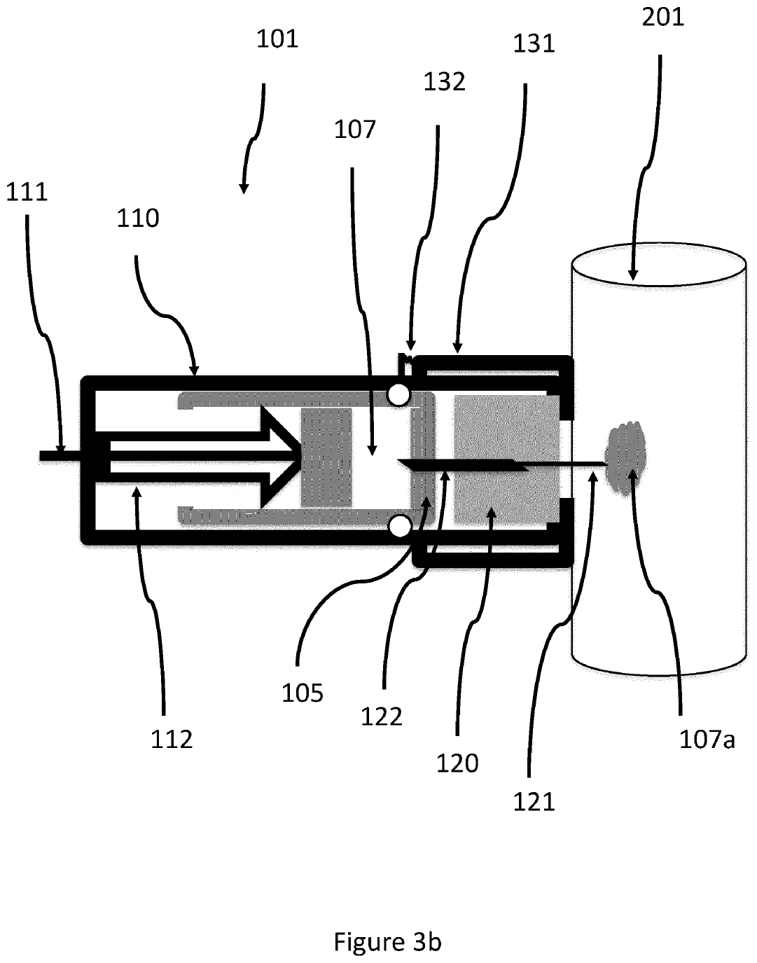

FIGS. 3a and 3b show the auto-injector 101 during injection. Auto-injector 101 is pressed against injection site 201. Needle 121 is inside the injection site 201 and delivering drug solution or suspension 107a as a bolus. Needle guard 131 has been pushed against injection site 201 and been pushed back relative to casing 110 against spring 132 pushing back shuttle 120. Needle 122 has pierced septum 105 and the pressurized contents 107 is flowing into injection site 201 via needles 121 and 122 and delivering a bolus 107a. The indicator 111 is moving forward showing injection progress. To ensure a complete injection, the injection needle penetrates the injection site (FIG. 3a) before the septum needle penetrates the septum (FIG. 3b).

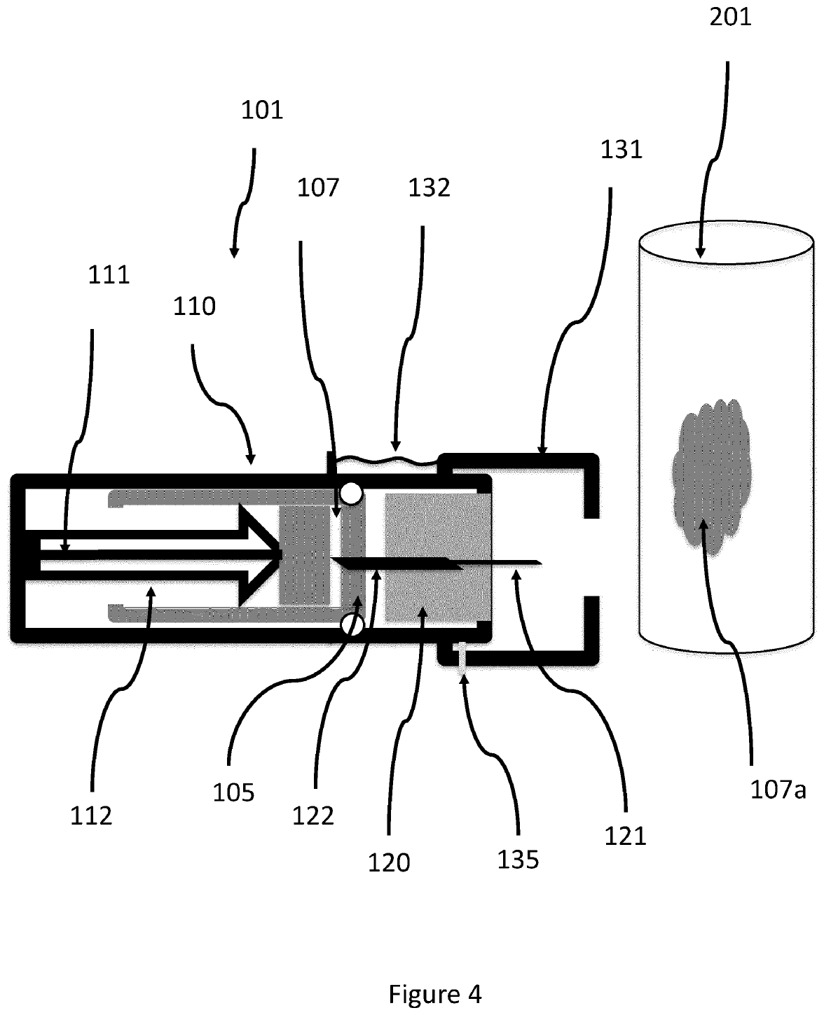

In FIG. 4 the auto injector 101 has finished injecting, the needle guard 131 has been pushed back by spring 132 and been locked into position by locking mechanism 135.

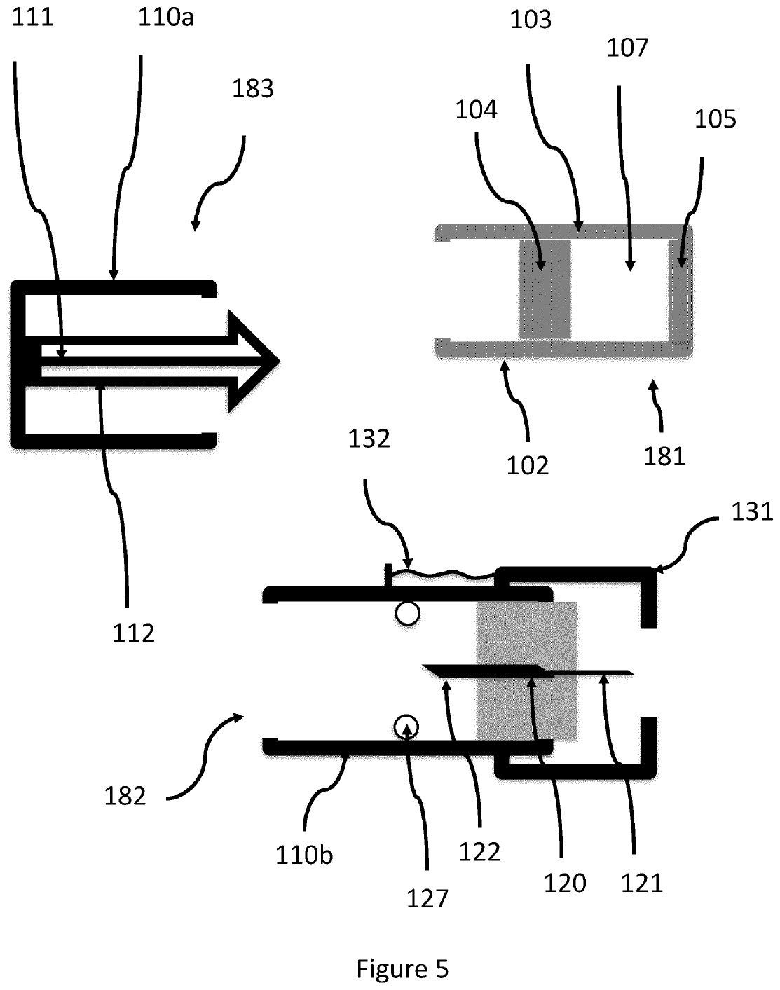

FIG. 5 shows a modular auto injector according to the present invention which is made up by three sub-assemblies: A cartridge sub-assembly 181, a needle and needle guard sub-assembly 182 and a power source and indicator sub-assembly 183.

Cartridge sub-assembly 181 has a cartridge 102 with barrel 103, drug volume 107 held by plunger 104 and septum 105.

Power source and indicator sub-assembly 183 has a biased means or spring 112 and indicator rod 111 held within casing 110a.

Needle and needle guard sub-assembly 182 has a casing 110b holding a needle shuttle 120 with needles 121 and 122. A needle guard 131 for preventing needle stick injuries is biased outwards by spring 132. Module 182 may be supplied sterilized. It also has a guard locking mechanism a shoulder 127 that retains the cartridge 102 in its correct position when inserted not shown.

Module 182 may contain a needle for subcutaneous, intramuscular or intra-dermal injections.

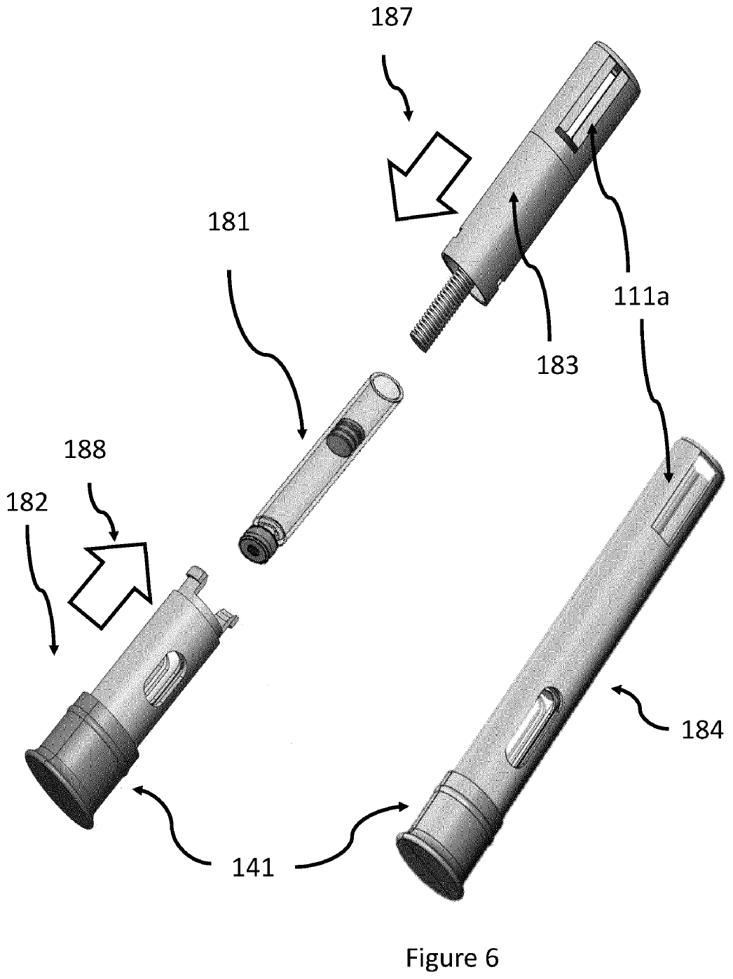

FIG. 6 shows a 3-dimensional view of the modular auto injector. Cartridge 181 is placed into sub-assembly 182 followed by sub-assemblies 183 and 182 being locked together in the directions of arrows 187 and 188 pressurizing the drug contents in cartridge 181.

The assembled auto injector 184 is shown with an additional indicator cap and indicator knob 111a which is attached to the indicator rod 111 (not shown) and an additional front cap 141 which are optional features.

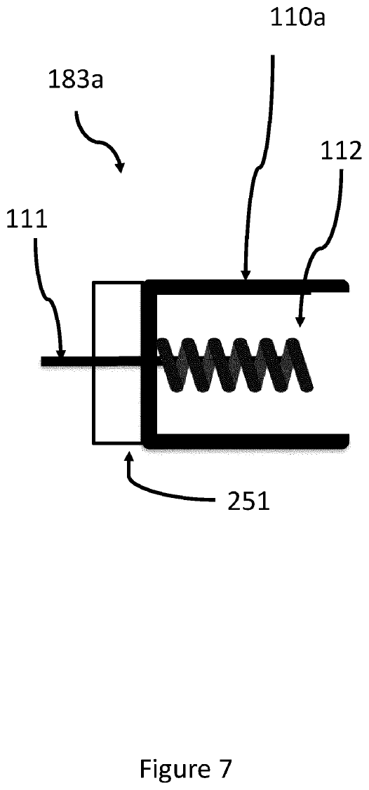

In FIG. 7 sub-assembly 183a has a locking member 251 holding spring 112 compressed and back by the indicator rod 111 for ease of device assembly. The locking member 251 is slowly removed after assembly of the device freeing spring 112 to slowly compress cartridge contents and avoid a pressure pulse. The locking member 251 may be a nut on a threaded indicator rod or any other mechanism.

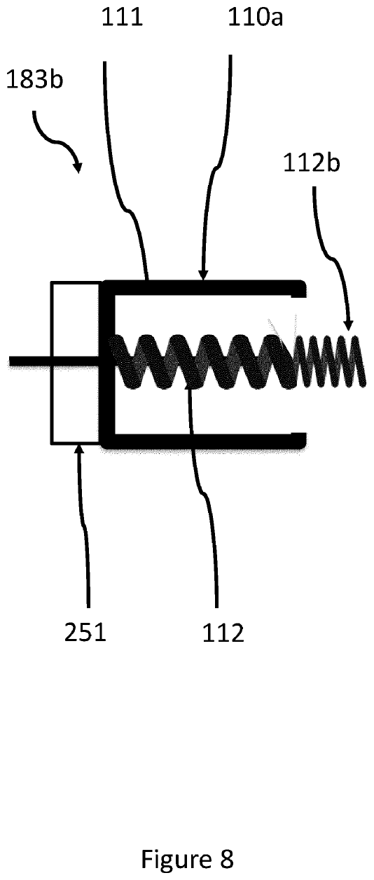

In FIG. 8 sub-assembly 183b has an additional spring 112b which is weaker than spring 112 that compresses the drug solution in the cartridge after the device is assembled. Before injecting the locking device 251 is removed so that spring 112 can fully pressurize the cartridge contents.

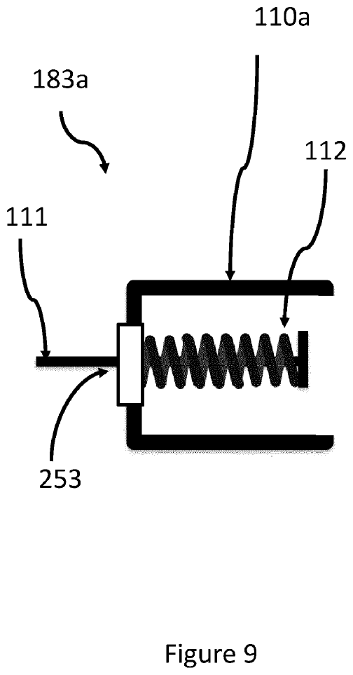

In FIG. 9 another embodiment is shown. A latch 253 holds the spring 112 compressed and holds it back for easy assembly. Latch 253 releases the spring when the device is assembled by outwards axial movement of the indicator 111 compressing the drug contents.

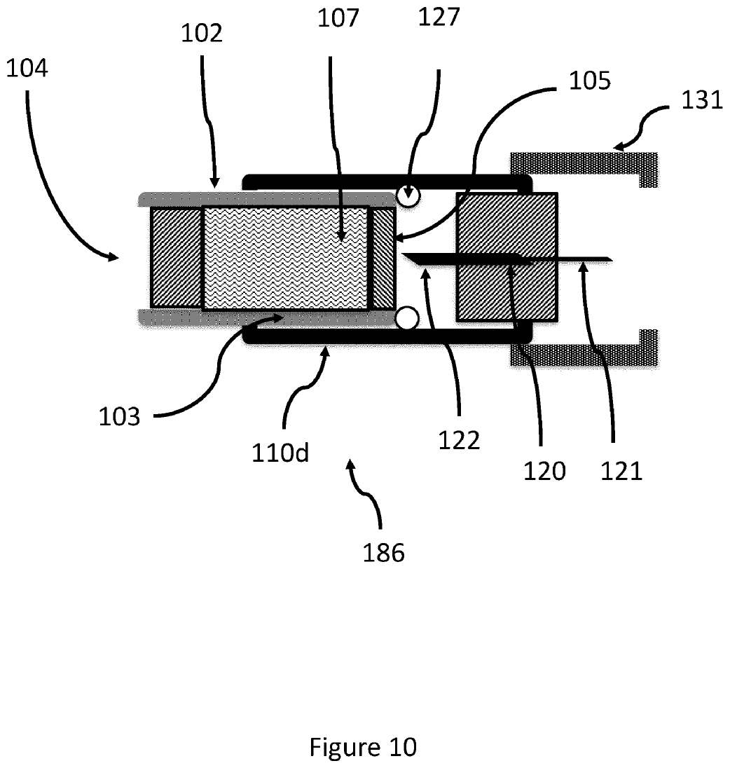

FIG. 10 shows another embodiment of the invention. Module 186 is a combination of sub-assemblies 181 and 182 and has the cartridge 102 supplied to the user with the needles 121 and 122 attached and is held in place with the needles 121 and 122.

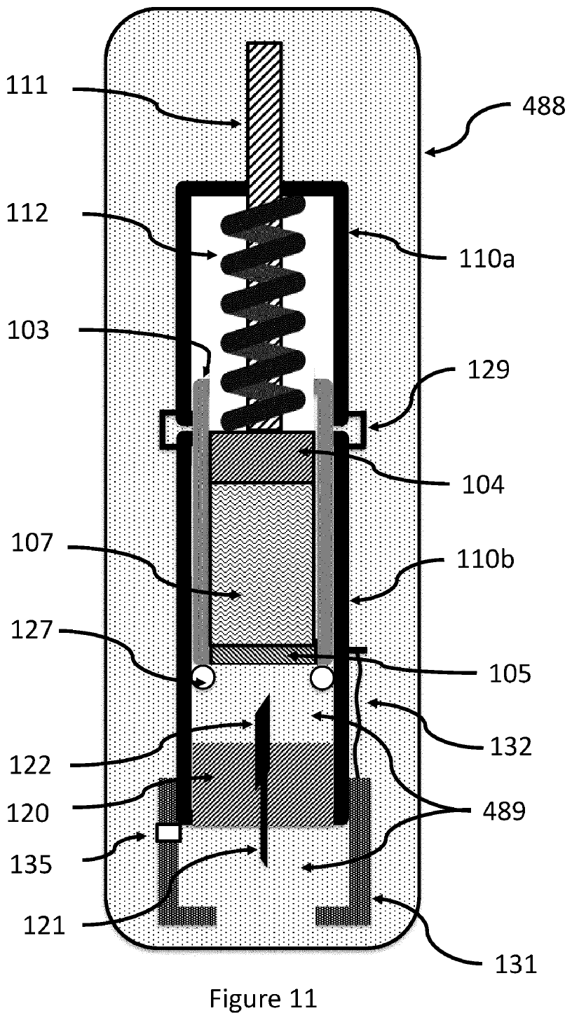

FIG. 11 shows an assembled and ready-to-use auto-injector that is being placed in a blister or pouch 488 and submitted to a gaseous environment for terminal sterilization, 489 indicates the sterilized area.

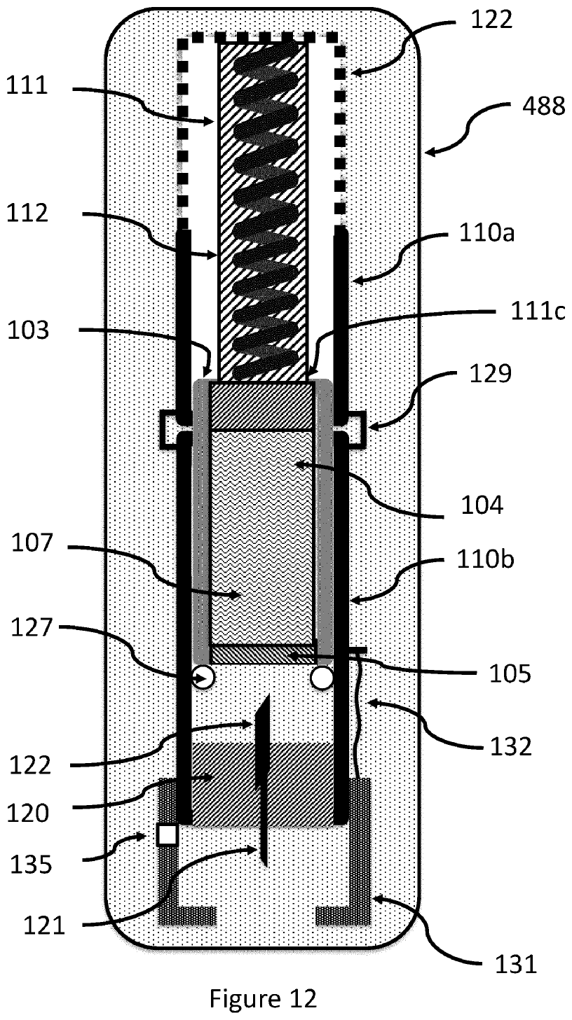

FIG. 12 shows an assembled and ready-to-use auto-injector wherein the indicator 111 contains the spring 112 within and an cap 122 that allows the observation of the injection progress.

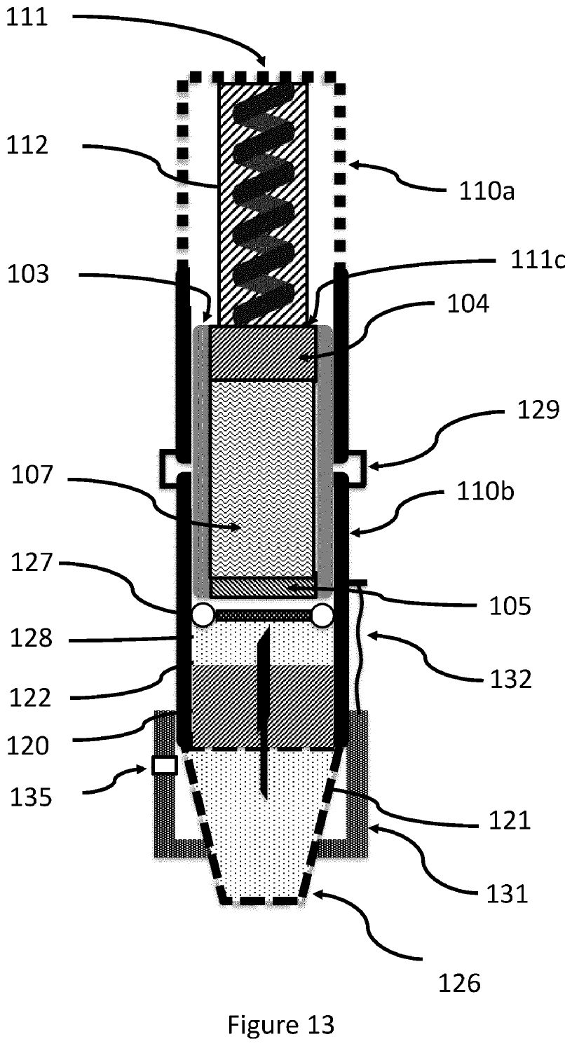

FIG. 13 shows an assembled and ready-to-use auto-injector comprising an additional seal 128 and a elastomeric needle cap 126 to keep the needles sterile. The use of cap 126 and optionally seal 128 allows for terminal sterilization without the use of a blister. For easier removal from needle guard 131 the cap 126 is made from an elastomeric material such as rubber.

A preferred embodiment of the present invention is a ready-to-use modular auto-injector that comprises
a cartridge sub-assembly 181 comprising a barrel 103, a drug volume 107 held by a movable plunger or stopper 104 and a crimped septum 105,
a power source and and indicator sub-assembly 183 comprising a rear casing 110a holding a biased means 112 and an indicator rod 111,
a needle and needle guard sub-assembly 182 comprising a front casing 110b holding a needle shuttle 120 with an injection needle 121 and a septum needle 122, a needle guard 131 and spring 132, a locking mechanism 135 that prevents the needle guard 131 from being pushed back a second time, and a means or shoulder 127 to retain the cartridge 102 in its correct position when the device is assembled, where the assembled device is placed in a blister or pouch 488 and terminally sterilized.

Another preferred embodiment of the present invention is a ready-to-use and re-usable modular auto-injector that comprises
a cartridge sub-assembly 181 comprising a barrel 103, a drug volume 107 held by a movable plunger or stopper 104 and a crimped septum or cap 105,
a and and indicator assembly 183 comprising a rear casing 110a n indicator rod 111 that contains a spring 112 within,
a needle and needle guard sub-assembly 182 comprising a front casing 110b holding a needle shuttle 120 with an injection needle 121 and a septum needle 122, a needle guard 131 and spring 132, a locking mechanism 135 that prevents the needle guard 131 from being pushed back a second time, a shoulder 127 to retain the cartridge 102 in its correct position when inserted and a seal 128 and an removable elastomeric needle cap 126 to keep the needles sterile, and
reversible connection means 129 allowing a re-assembly or user assembly of the device,
where assembly 182 is supplied sterilized and ready for use either in a blister and/or supplied with a seal 128 and an needle cap 126.

1. An auto-injector comprising
an outer casing holding a standard cartridge consisting of a barrel with a drug contents being contained by a movable plunger at one end and a seal and metal crimp at the other end,
a biased means that pushes against the movable plunger of the cartridge pressurizing the drug contents,
a needle shuttle being held within the casing comprising two needles that are attached and connected to each other, a first needle being the injection needle and a second needle being the septum needle,
a means to retain the cartridge in its correct position when inserted,
a needle guard that is held to the outer casing, being biased outwards such that the needle is always covered by the needle guard.

2. A auto-injector according to claim 1, wherein the auto-injector further comprising a locking mechanism that prevents the needle guard from being pushed back a second time.

3. A auto-injector according to claim 1, wherein the auto-injector further comprising an indicator rod that is attached to the movable plunger.

4. A auto-injector according to claim 3, wherein the indicator rod has a closed end containing the biased means in form of a spring within, the outer casing comprising a transparent portion that allows to monitor the progress and status of the injection progress.

5. A modular auto-injector according to claim 1, wherein the modular auto-injector comprises three sub-assemblies, namely
a cartridge sub-assembly,
a power source and indicator assembly, and
a needle and needle guard assembly.

6. Modular auto-injector according to claim 5 wherein the cartridge sub-assembly comprises a barrel and a drug volume being bounded by a plunger and a crimped septum.

7. Modular auto-injector according to claim 5 wherein the power source and indicator sub-assembly comprises a biased means and an indicator rod that is held within a rear casing.

8. Modular auto-injector according to claim 7, wherein the indicator rod may be connected to the movable plunger in a way that it moves with it.

9. Modular auto-injector according to claim 7, wherein the biased means of the power source and indicator sub-assembly is a spring that further comprises a spring locking member.

10. Modular auto-injector according to claim 9, wherein the power source and indicator sub-assembly further comprises an additional spring which is weaker than the spring to pressurise the cartridge drug contents prior to injection while the spring is locked by the spring locking member.

11. Modular auto-injector according to claim 5, wherein the needle and needle guard sub-assembly comprises a casing holding a needle shuttle with an injection needle and a septum needle, a needle guard and spring, a locking mechanism that prevents the needle guard from being pushed back a second time, and a shoulder to retain the cartridge in its correct position when inserted.

12. Modular auto-injector according to claim 11 for re-assembly, wherein the needle and needle guard sub-assembly further comprises a seal and a needle cap, the biased means of the power source and indicator sub-assembly is an electric motor or a liquified gas canister or a spring with means to re-compress the spring, and the needle and needle guard sub-assembly and the power source and indicator sub-assembly are reversibly connected by a a bayonet fit or a screw or are hinged together.

13. Process for the assembly of a modular auto-injector according to claim 5 including the following steps:
inserting a cartridge into a needle and needle guard sub-assembly, and
connecting a power source and indicator sub-assembly via connection means to the needle and needle guard sub-assembly with inserted cartridge in a way that the drug contents in the cartridge is pressurized.

14. Process according to claim 13, further comprising the steps of
inserting the pre-assembled device in a blister or pouch, and
submitting the blister or pouch to terminal sterilization.

15. A assembly process according to claim 14, wherein the terminal sterilization is carried out by ethylene oxide or nitrogen dioxide.
